(19) United States
(12) Patent Application Publication
(10) Pub. No.: US 2023/0263470 A1
(43) Pub. Date: Aug. 24, 2023

(54) Medical Adhesive Patch Assembly

(71) Applicant: Nickey Neilsen, Simi Valley, CA (US)

(72) Inventor: Nickey Neilsen, Simi Valley, CA (US)

(21) Appl. No.: 17/678,816

(22) Filed: Feb. 23, 2022

(51) Int. Cl. A61B 5/00; A61B 5/145

(52) U.S. Cl. CPC A61B 5/6833; A61B 5/14532; A61B 5/68335

(57) ABSTRACT

A medical adhesive patch assembly for attaching a glucose monitor to a user's skin includes a patch is continuously arcuate such that the patch forms a closed ring. The closed ring defined by the patch is elongated along a first axis such that the patch has an ovoid shape thereby facilitating the patch to be positioned around a skin mounted glucose monitor. An adhesive layer is bonded to the patch to adhere to a user's skin. The adhesive layer is comprised of a hypoallergenic material to inhibit irritating the user's skin. A protective sheet is removably positioned on the adhesive layer for inhibiting the adhesive layer from being adhered to an object. The protective sheet is peelable from the adhesive layer to expose the adhesive layer.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to adhesive patch devices and more particularly pertains to a new adhesive patch device for retaining a glucose monitor on a user's skin for at least ten days. The device includes a patch which forms a closed ring for positioning around a glucose monitor. The device includes an adhesive layer that is bonded to the patch. The adhesive layer is water resistant and the adhesive layer has a service life of at least ten days.

### (2) Description of Related Art Including Information Disclosed Under 37 CFR 1.97 and 1.98

The prior art relates to adhesive patch devices including a variety of modular patch devices for attaching a sensing device and which has electrical contacts integrated therein. The prior art discloses a bandage device that includes a lower bandage and an upper bandage which forms a closed loop and which is positionable over the lower bandage. The prior art discloses a template sheet which can be adhered to a user's skin for making the user's skin. The prior art discloses a wearable electronic device that includes a lower adhesive patch, a plurality of electronic sensors attached to the lower adhesive patch and an upper adhesive patch that can be adhered to the lower adhesive patch.

### BRIEF SUMMARY OF THE INVENTION

An embodiment of the disclosure meets the needs presented above by generally comprising a patch is continuously arcuate such that the patch forms a closed ring. The closed ring defined by the patch is elongated along a first axis such that the patch has an ovoid shape thereby facilitating the patch to be positioned around a skin mounted glucose monitor. An adhesive layer is bonded to the patch to adhere to a user's skin. The adhesive layer is comprised of a hypoallergenic material to inhibit irritating the user's skin. A protective sheet is removably positioned on the adhesive layer for inhibiting the adhesive layer from being adhered to an object. The protective sheet is peelable from the adhesive layer to expose the adhesive layer.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a medical adhesive patch assembly according to an embodiment of the disclosure.

FIG. 2 is a back perspective view of an embodiment of the disclosure.

FIG. 3 is a back view of an embodiment of the disclosure.

FIG. 4 is a front view of an alternative embodiment of the disclosure.

FIG. 5 is an exploded in-use view of an embodiment of the disclosure.

FIG. 6 is a perspective in-use view of an embodiment of the disclosure.

FIG. 7 is a right side view of an embodiment of the disclosure.

### DETAILED DESCRIPTION OF THE INVENTION

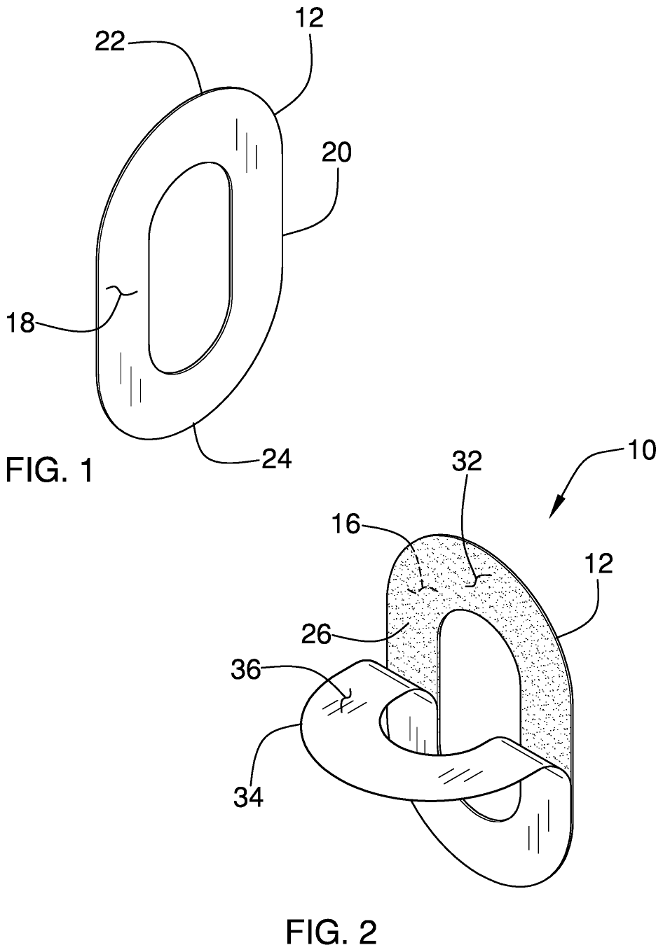

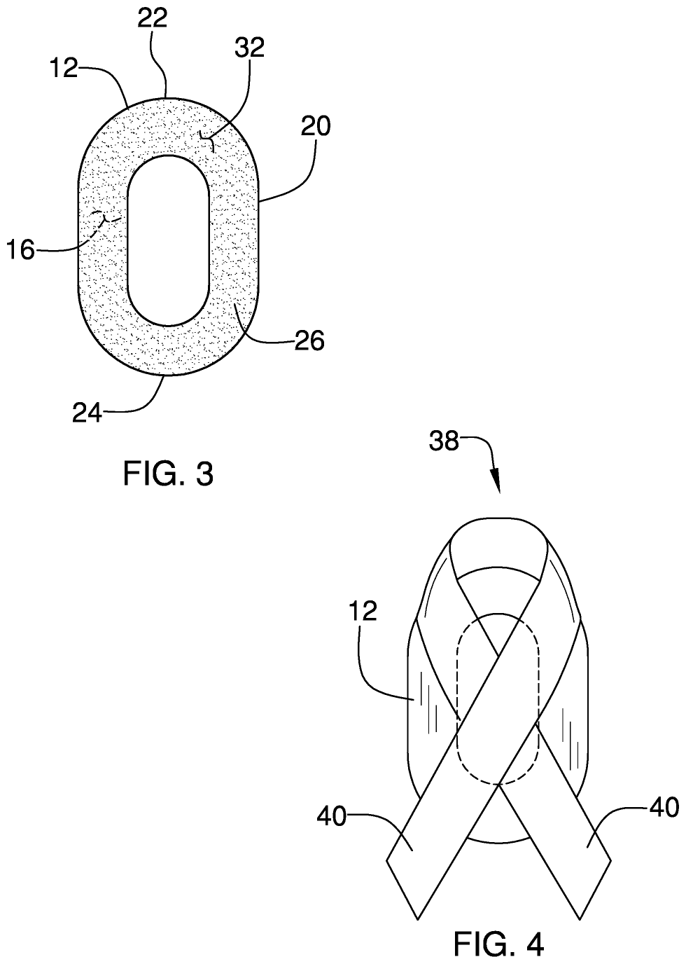

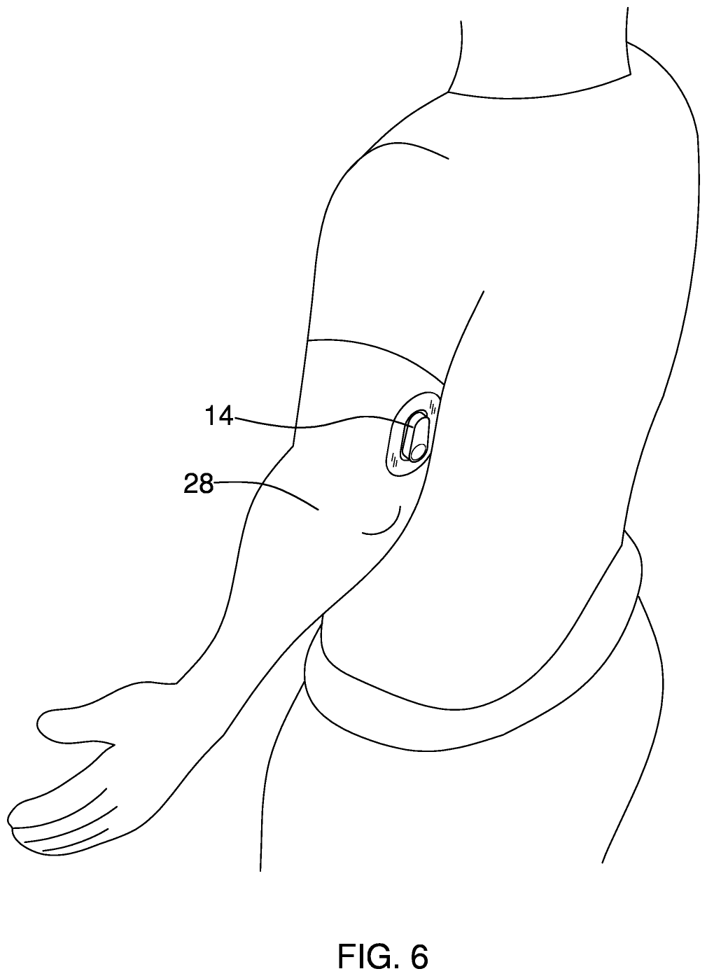

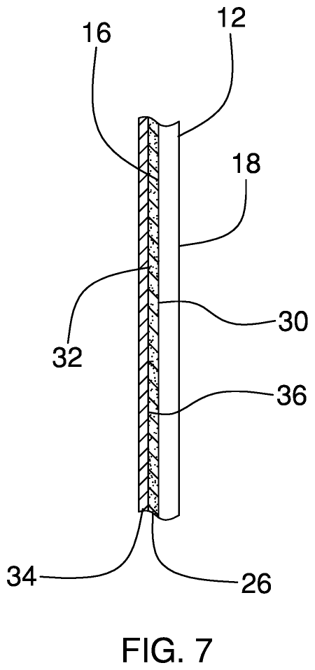

With reference now to the drawings, and in particular to FIGS. 1 through 7 thereof, a new adhesive patch device embodying the principles and concepts of an embodiment of the disclosure and generally designated by the reference numeral 10 will be described.

As best illustrated in FIGS. 1 through 7, the medical adhesive patch assembly 10 generally comprises a patch 12 that is continuously arcuate such that the patch 12 forms a closed ring. The closed ring defined by the patch 12 is elongated along a first axis such that the patch 12 has an ovoid shape. In this way the patch 12 can be positioned around a skin mounted glucose monitor 14. The skin mounted glucose monitor 14 may be a Continuous Glucose monitoring device of any conventional design employed by diabetics to monitor blood sugar levels. The patch 12 has a first surface 16, a second surface 18 and a perimeter edge 20 extending between the first surface 16 and the second surface 18. The perimeter edge 20 has a top side 22 and a bottom side 24, and the ring formed by the patch 12 is elongated between the top side 22 and the bottom side 24.

An adhesive layer 26 is bonded to the patch 12 to adhere to a user's skin 28 thereby facilitating the glucose monitor 14 to be adhered to the user's skin 28. The adhesive layer 26 is comprised of a hypoallergenic material to inhibit irritating the user's skin 28. The adhesive layer 26 comprises a water resistant adhesive to resist being dislodged from the user's skin 28 by sweat or water. In this way the adhesive layer 26 will remain in place on an athletically active user, for example, or other user that is prone to excessive perspiration. The adhesive layer 26 has a service life of at least 240.0 hours thereby facilitating the adhesive layer 26 to retain the skin mounted glucose monitor 14 in place for at least 10.0 days.

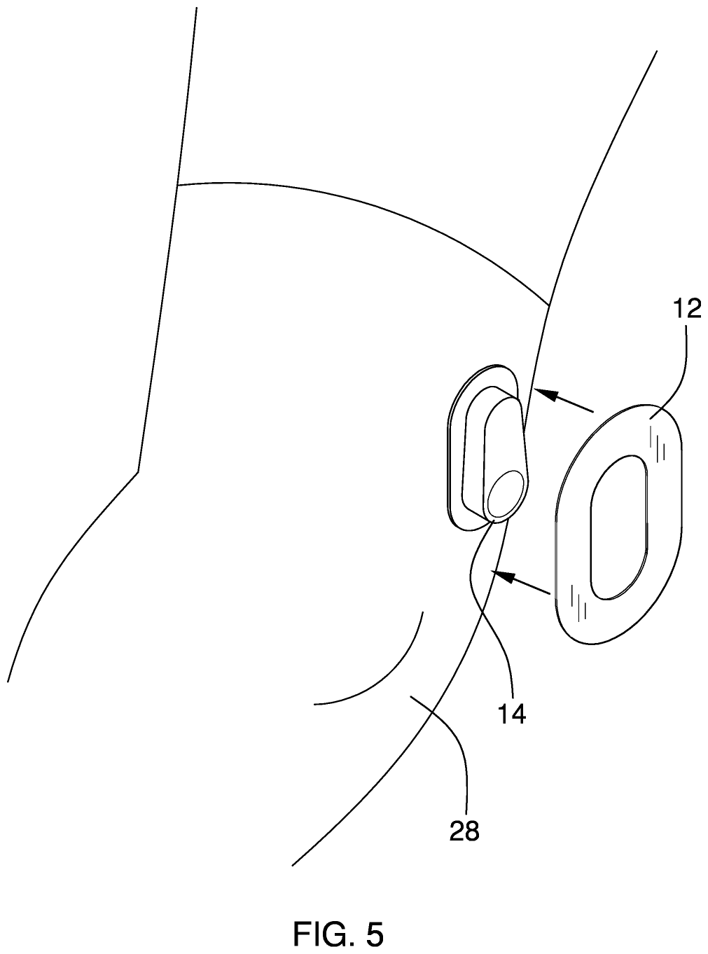

The adhesive layer 26 has a primary surface 30 and a secondary surface 32, and the primary surface 30 is bonded to the first surface 16 of the patch 12 such that the adhesive layer 26 completely covers the first surface 16. A protective sheet 34 is removably positioned on the adhesive layer 26 for inhibiting the adhesive layer 26 from being adhered to an object. The protective sheet 34 is peelable from the adhesive layer 26 to expose the adhesive layer 26 and the protective sheet 34 forms a closed loop that is elongated to match the shape of the patch 12. The protective sheet 34 has a top surface 36 and the top surface 36 is positioned on the secondary surface 32 of the adhesive layer 26 having the protective sheet 34 completely covering the adhesive layer 26. The top surface 36 comprises a non adherent material thereby inhibiting the top surface 36 from being adhered to the secondary surface 32. In an alternative embodiment 38 shown in FIG. 4, the patch 12 has a pair of arms 40 each extending laterally away from the top side 22 of the perimeter edge 20 of the patch 12. Each of the arms 40 is foldable across an opening 42 of the ring defined by the patch 12 thereby facilitating the arms 40 to extend over the glucose monitor 14.

In use, the glucose monitor 14 is positioned at a desired location on the user's body to facilitate the glucose monitor 14 to monitor the user's blood sugar. The protective sheet 34 is removed from the adhesive layer 26 and the patch 12 is positioned around the glucose monitor 14 such that the adhesive layer 26 adheres to the user's skin 28. In this way the patch 12 secures the glucose monitor 14 in place. Furthermore, the adhesive layer 26 ensures that the glucose monitor 14 remains in place for at least 10.0 days. In this way the glucose monitor 14 does not have to be replaced before the glucose monitor 14 expires.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medical adhesive patch assembly for retaining a glucose monitor on a user's skin for a minimum of ten days, said assembly comprising:
a patch being continuously arcuate such that said patch forms a closed ring, said closed ring defined by said patch being elongated along a first axis such that said patch has an ovoid shape thereby facilitating said patch to be positioned around a skin mounted glucose monitor;
an adhesive layer being bonded to said patch wherein said adhesive layer is configured to adhere to a user's skin, said adhesive layer being comprised of a hypoallergenic material wherein said adhesive layer is configured to inhibit irritating the user's skin; and
a protective sheet being removably positioned on said adhesive layer for inhibiting said adhesive layer from being adhered to an object, said protective sheet being peelable from said adhesive layer to expose said adhesive layer.

2. The assembly according to claim 1, wherein said patch has a first surface, a second surface and a perimeter edge extending between said first surface and said second surface, said perimeter edge having a top side and a bottom side, said ring formed by said patch being elongated between said top side and said bottom side.

3. The assembly according to claim 1, wherein said adhesive layer comprises a water resistant adhesive wherein said adhesive layer is configured to resist being dislodged from the user's skin by sweat or water.

4. The assembly according to claim 1, wherein said adhesive layer has a service life of at least 240.0 hours thereby facilitating said adhesive layer to retain the skin mounted glucose monitor in place for at least 10.0 days.

5. The assembly according to claim 2, wherein said adhesive layer has a primary surface and a secondary surface, said primary surface being bonded to said first surface of said patch having said adhesive layer completely covering said first surface.

6. The assembly according to claim 1, wherein said protective sheet forms a closed loop being elongated to match the shape of said patch.

7. The assembly according to claim 5, wherein said protective sheet has a top surface, said top surface being positioned on said secondary surface of said adhesive layer having said protective sheet completely covering said adhesive layer, said top surface comprising a non adherent material thereby inhibiting said top surface from being adhered to said secondary surface.

8. A medical adhesive patch assembly for retaining a glucose monitor on a user's skin for a minimum of ten days, said assembly comprising:
a patch being continuously arcuate such that said patch forms a closed ring, said closed ring defined by said patch being elongated along a first axis such that said patch has an ovoid shape thereby facilitating said patch to be positioned around a skin mounted glucose monitor, said patch having a first surface, a second surface and a perimeter edge extending between said first surface and said second surface, said perimeter edge having a top side and a bottom side, said ring formed by said patch being elongated between said top side and said bottom side;
an adhesive layer being bonded to said patch wherein said adhesive layer is configured to adhere to a user's skin thereby facilitating said glucose monitor to be adhered to the user's skin, said adhesive layer being comprised of a hypoallergenic material wherein said adhesive layer is configured to inhibit irritating the user's skin, said adhesive layer comprising a water resistant adhesive wherein said adhesive layer is configured to resist being dislodged from the user's skin by sweat or water, said adhesive layer having a service life of at least 240.0 hours thereby facilitating said adhesive layer to retain the skin mounted glucose monitor in place for at least 10.0 days, said adhesive layer having a primary surface and a secondary surface, said primary surface being bonded to said first surface of said patch having said adhesive layer completely covering said first surface; and
a protective sheet being removably positioned on said adhesive layer for inhibiting said adhesive layer from being adhered to an object, said protective sheet being peelable from said adhesive layer to expose said adhesive layer, said protective sheet forming a closed loop being elongated to match the shape of said patch, said protective sheet having a top surface, said top surface being positioned on said secondary surface of said adhesive layer having said protective sheet completely covering said adhesive layer, said top surface comprising a non adherent material thereby inhibiting said top surface from being adhered to said secondary surface.

9. The assembly according to claim 8, wherein said patch has a pair of arms each extending laterally away from said top side of said perimeter edge of said patch, each of said arms being foldable across an opening of said ring defined by said patch thereby facilitating said arms to extend over the glucose monitor.

10. A medical adhesive patch system for retaining a glucose monitor on a user's skin for a minimum of ten days, said assembly comprising:
a glucose monitor being fixable to a user's skin wherein said glucose monitor is configured to monitor the user's glucose levels;
a patch being continuously arcuate such that said patch forms a closed ring, said closed ring defined by said patch being elongated along a first axis such that said patch has an ovoid shape thereby facilitating said patch to be positioned around said glucose monitor, said patch having a first surface, a second surface and a perimeter edge extending between said first surface and said second surface, said perimeter edge having a top side and a bottom side, said ring formed by said patch being elongated between said top side and said bottom side;
an adhesive layer being bonded to said patch for adhering to a user's skin thereby securing said glucose monitor to the user's skin, said adhesive layer being comprised of a hypoallergenic material wherein said adhesive layer is configured to inhibit irritating the user's skin, said adhesive layer comprising a water resistant adhesive wherein said adhesive layer is configured to resist being dislodged from the user's skin by sweat or water, said adhesive layer having a service life of at least 240.0 hours thereby facilitating said adhesive layer to retain the skin mounted glucose monitor in place for at least 10.0 days, said adhesive layer having a primary surface and a secondary surface, said primary surface being bonded to said first surface of said patch having said adhesive layer completely covering said first surface; and
a protective sheet being removably positioned on said adhesive layer for inhibiting said adhesive layer from being adhered to an object, said protective sheet being peelable from said adhesive layer to expose said adhesive layer, said protective sheet forming a closed loop being elongated to match the shape of said patch, said protective sheet having a top surface, said top surface being positioned on said secondary surface of said adhesive layer having said protective sheet completely covering said adhesive layer, said top surface comprising a non adherent material thereby inhibiting said top surface from being adhered to said secondary surface.
